(19) United States
(12) Patent Application Publication
(10) Pub. No.: US 2022/0333146 A1
(43) Pub. Date: Oct. 20, 2022

(54) SYNTHESIS OF POLYNUCLEOTIDE BOTTLEBRUSH POLYMER

(71) Applicant: ILLUMINA SINGAPORE PTE. LTD., Singapore (SG)

(72) Inventors: Xiangyuan YANG, Singapore (SG); Saurabh NIRANTAR, Singapore (SG); Yin Nah TEO, Singapore (SG)

(73) Assignee: ILLUMINA SINGAPORE PTE. LTD., Singapore (SG)

(21) Appl. No.: 17/719,899

(22) Filed: Apr. 13, 2022

Related U.S. Application Data
(60) Provisional application No. 63/174,768, filed on Apr. 14, 2021.

(51) Int. Cl. C12P 19/34; C12N 9/12

(52) U.S. Cl. CPC C12P 19/34; C12N 9/1264; C12Y 207/07031; C08G 83/002

(57) ABSTRACT

Provided is a method including extending a ssDNA by sequentially adding a plurality of modified nucleoside triphosphates to the ssDNA, wherein the base of the modified nucleoside triphosphates includes a primary modification selected from (i) a primary polynucleotide attached to the base of the modified nucleoside triphosphate, and (ii) a site on the base for covalent attachment of a primary polynucleotide to the base, further comprising covalently attaching a primary polynucleotide to the base after the polymerizing.

### CROSS-REFERENCE TO RELATED APPLICATIONS

This application claims benefit of priority from U.S. Provisional Patent Application No. 63/174,768, filed Apr. 14, 2021, the entire contents of which is incorporated herein by reference in its entirety.

### SEQUENCE LISTING

The instant application contains a Sequence Listing, created on Mar. 30, 2022; the file, in ASCII format, is designated H2301932.txt and is 4.6 KB in size. The file is hereby incorporated by reference in its entirety into the instant application.

### BACKGROUND

Polynucleotides, such as polymers including DNA polynucleotides, are useful structures adaptable for various applications. The nanoscale architecture of DNA-based structures renders them attractive for technologies requiring assembly of sub-micron features with predetermined design characteristics, shapes, sizes, etc. DNA-based polynucleotides having, for example, branched or bottlebrush polymer architectures may find uses in numerous technology area but methods for their synthesis are lacking. The present disclosure is directed to overcoming these and other deficiencies in the art.

### SUMMARY

In an aspect, provided is a method, including extending a ssDNA by sequentially adding a plurality of modified nucleoside triphosphates to the ssDNA, wherein the base of the modified nucleoside triphosphates includes a primary modification and the primary modification is selected from (i) a primary polynucleotide attached to the base of the modified nucleoside triphosphate, and (ii) a site on the base for attachment of a primary polynucleotide to the base, further including attaching a primary polynucleotide to the base after the polymerizing.

In another example, the polymerase is a terminal deoxynucleotidyl transferase (TdT).

In yet another example, the TdT includes an amino acid sequence that is at least 80% identical to SEQ ID NO: 1. In another example, the TdT includes an amino acid sequence of SEQ ID NO: 1.

In a further example, a ratio of (a) a number of nucleoside triphosphates including the primary modification added to the ssDNA to (b) a number of nucleoside triphosphates not including a primary modification added to the ssDNA is from 1:100 to 100:1.

In yet a further example, the polymerase is a template-dependent polymerase.

In still another example, a template for the polymerase includes a ratio of (a) a number of nucleotides complementary to nucleoside triphosphates including primary modifications to (b) a number of nucleotides complementary to nucleoside triphosphates not including primary modifications, and the ratio is from 1:100 to 100:1.

In still a further example, the primary polynucleotides further include one or more nucleotide including a secondary modification, wherein the secondary modification includes a site on a base for attachment of a secondary polynucleotide side chain. In an example, the primary polynucleotides further includes one or more nucleotide comprising a secondary modification, wherein the secondary modification comprises a site on a base for covalent attachment of a secondary polynucleotide side chain.

Another example further includes attaching a secondary polynucleotide to one or more nucleotide of the primary polynucleotides including a secondary modification. Still another example further includes covalently attaching a secondary polynucleotide to one or more nucleotide of the primary polynucleotides comprising a secondary modification.

In another example, the primary modification includes a primary polynucleotide covalently attached to the base of the modified nucleoside triphosphate. In another example, the primary modification includes a primary polynucleotide attached to the base of the modified nucleoside triphosphate, wherein the primary polynucleotide is attached to the base of the modified nucleoside triphosphate by a covalent bond selected from an amine-NETS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

In yet another example, the primary modification includes a site on the base for attachment of a primary polynucleotide to the base, wherein the site on the base for attachment of a primary nucleotide to the base is for covalent attachment. In still another example, the primary modification includes a site on the base for attachment of a primary polynucleotide to the base, and the further including attaching a primary polynucleotide to the base after the polymerizing includes forming a covalent bond selected from an amine-NETS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

In a further example, the site on a base for covalent attachment of a secondary polynucleotide is a site for attachment by a covalent bond selected from an amine-NETS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

In yet a further example, covalently attaching a secondary polynucleotide to one or more nucleotides of the primary polynucleotides including a secondary modification includes forming a covalent bond selected from an amine-NETS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne bond, and an azide-norbornene bond.

### BRIEF DESCRIPTION OF THE DRAWINGS

These and other features, aspects, and advantages of the present disclosure will become better understood when the following detailed description is read with reference to the accompanying drawings, wherein:

FIG. 1 shows an example of method of synthesizing a polynucleotide bottlebrush polymer in accordance with aspects of this disclosure.

FIG. 2 shows another example of method of synthesizing a polynucleotide bottlebrush polymer in accordance with aspects of this disclosure.

FIG. 3 shows another example of method of synthesizing a polynucleotide bottlebrush polymer in accordance with aspects of this disclosure.

FIG. 4 shows another example of method of synthesizing a polynucleotide bottlebrush polymer in accordance with aspects of this disclosure.

FIG. 5 shows another example of method of synthesizing a polynucleotide bottlebrush polymer in accordance with aspects of this disclosure.

FIGS. 6 and 7 another example of method of synthesizing a polynucleotide bottlebrush polymer in accordance with aspects of this disclosure.

FIG. 8 shows another example of method of synthesizing a polynucleotide bottlebrush polymer in accordance with aspects of this disclosure.

FIG. 9 shows another example of method of synthesizing a polynucleotide bottlebrush polymer in accordance with aspects of this disclosure.

### DETAILED DESCRIPTION

This disclosure relates to a method for synthesizing a polymer including a polynucleotide bottlebrush polymer structure. A polynucleotide bottlebrush polymer includes a substantially linear polynucleotide backbone, including a sequence of nucleotides connected to one another by phosphodiester bonds (e.g., a phosphodiester bond included in a connection between a 5′ carbon of a ribose of one nucleotide monomer and a 3′ carbon or a ribose of an adjacent nucleotide monomer). As disclosed herein, a polynucleotide bottlebrush polymer further includes one or more primary polynucleotide side chain. A primary polynucleotide side chain may be connected to a nucleobase of a nucleotide of the polynucleotide backbone by one or more covalent bond or by one or more non-covalent bond. Nucleobases of a plurality of nucleotides of the polynucleotide backbone may be connected to primary polynucleotide side chains, to form a polynucleotide bottlebrush polymer.

In an example as disclosed herein, a polynucleotide bottlebrush polymer may further include one or more secondary polynucleotide side chain. A secondary polynucleotide side chain may be connected to a nucleobase of a nucleotide of a primary polynucleotide side chain by one or more covalent bond or one or more non-covalent bond. Nucleobases of a plurality of nucleotides of a polynucleotide side chain may be connected to a secondary polynucleotide side chain. Nucleobases of a nucleotide of a plurality of polynucleotide side chain may be connected to a secondary polynucleotide side chain. And nucleobases of a plurality of nucleotides of a plurality of polynucleotide side chain may be connected to a secondary polynucleotide side chain.

In further examples, a next generation of polynucleotide side chains may be added to a previously added generation of polynucleotide side chains, beyond secondary polynucleotide side chains. For example, similar to secondary polynucleotide side chains extending from modified nucleobases of a nucleotide of a primary polynucleotide side chain, a polymer may include tertiary polynucleotide side chains extending from modified nucleobases of nucleotides of secondary polynucleotide side chains. A polymer may further include quaternary polynucleotide side chains extending from modified nucleobases of nucleotides of tertiary polynucleotide side chains. As an extension of this pattern, sequential generations of polynucleotide side chains may be continued. For example, a polymer may include an nth generation of polynucleotide side chains extending from modified nucleobases of nucleotides of polynucleotide side chains of the (n−1)th generation, wherein polynucleotides of an nth generation are attached to modified nucleobases of nucleotides of an (n−1)th generation of polynucleotide side chains.

For example, a polymer may include secondary polynucleotide side chains (nth generation polynucleotide side chains) attached to modified nucleobases of nucleotides of primary polynucleotide side chains ((n−1)nth generation polynucleotide side chains). Such a polymer would have n generations of polynucleotide side chains and n would be 2. Or a polymer may include tertiary polynucleotide side chains (nth generation polynucleotide side chains) attached to modified nucleobases of nucleotides of secondary polynucleotide side chains ((n−1)th generation polynucleotide side chains), which secondary polynucleotide side chains would be attached to modified nucleobases of nucleotides of primary polynucleotide side chains ((n−2)th generation polynucleotide side chains). Such a polymer would have n generations of polynucleotide side chains and n would be 3. Or a polymer may include quaternary polynucleotide side chains (nth generation polynucleotide side chains) attached to modified nucleobases of nucleotides of tertiary polynucleotide side chains ((n−1)th generation polynucleotide side chains), which tertiary polynucleotide side chains would be attached to modified nucleobases of nucleotides of secondary polynucleotide side chains ((n−2)th generation polynucleotide side chains), which secondary polynucleotide side chains would be attached to modified nucleobases of nucleotides of primary polynucleotide side chains ((n−3)th generation polynucleotide side chains). Such a polymer would have n generations of polynucleotide side chains and n would be 4. A polymer may have n generations of polynucleotide side chains, in which n may be 1, 2, 3, 4, 5, 6, 7, 8, 9, or 10.

Any example disclosed herein of attaching or bonding a secondary polynucleotide side chain to a modified nucleobase of a nucleotide of a primary polynucleotide side chain may be used for attaching or bonding a polynucleotide side chain of an nth generation to a modified nucleobase of a nucleotide of a polynucleotide side chain of an (n−1)th generation. In an example, a first type of attachment may be used to attach a generation of polynucleotide side chains to polynucleotide side chains of a generation already included in a polymer, and a second type of attachment may subsequently be used for attaching a subsequent generation of polynucleotide side chains to polynucleotide side chains attached to the polymer by the first type of attachment. In an example, subsequently, a third type of attachment may be used for attaching a next generation of polynucleotide side chains.

Different types of attachment chemistries or attachment moieties as disclosed herein may be used in attaching successive generations of polynucleotide side chains. A nucleotide of a generation of polynucleotide side chains may include a modification capable of attaching to a polynucleotide of the next generation of polynucleotide side chains but not one or more generation of polynucleotide side chains subsequent thereto. And a polynucleotide side chain may include a modification or attachment moiety as disclosed herein for forming an attachment to a nucleotide of a polynucleotide of one previously added generation but not another or any other previously added generation. In an example, such differences may be such that a polynucleotide side chain of a generation may form an attachment with a modified nucleobase of a nucleotide of one generation but not with a modified nucleobase of a nucleotide of another or any other generation. For example, a secondary polynucleotide side chain may include a moiety for forming an attachment with an attachment moiety of a modified nucleobase of a nucleotide of a primary polynucleotide side chain, whereas a tertiary side chain, quaternary side chain, or side chain of another generation may not include a moiety for forming an attachment with an attachment moiety of a modified nucleobase of a nucleotide of a primary polynucleotide side chain. For example, a polynucleotide for attachment to a polymer as a polynucleotide side chain may have an attachment moiety for attaching to an attachment moiety of a modified nucleobase of a nucleotide of a polynucleotide backbone or of a polynucleotide side chain already included in the polymer but not for forming an attachment to a modified nucleobase of one of its own nucleotides.

A polynucleotide in a polynucleotide bottlebrush polymer as disclosed herein may be covalently attached to a nucleobase of a nucleotide. For example, a nucleotide may include a chemical modification of its nucleobase to include any chemical moiety amenable for formation of a covalent bond with another chemical moiety, which other chemical moiety may be present on the polynucleotide for attachment thereto. Various examples of complementary moieties for such attachments may be adopted, such as various pairs of moieties available for use in click chemistry reactions as further disclosed herein. A modification may be attached to a nucleobase by a linker.

A polynucleotide in a polynucleotide bottlebrush polymer as disclosed herein may be non-covalently attached to a nucleobase of a nucleotide. For example, a nucleotide may include a modification of its nucleobase to include a moiety amenable for formation of a non-covalent attachment with another moiety, which other moiety may be present on the polynucleotide for attachment thereto. Various examples of complementary moieties for such attachments may be adopted. For example, any examples of non-covalent attachment disclosed herein, in whatever context, is explicitly included as an example of a non-covalent attachment of a polynucleotide to a modified nucleobase of a polynucleotide of a polymer. For example, non-covalent protein-protein attachment may be used, such as avidin-biotin attachment or coiled-coil attachment, examples of which are disclosed herein as non-limiting examples of non-covalent attachments of a polynucleotide side chain to a modified nucleobase of a polymer.

In another example, a modified nucleobase of a nucleotide of a polynucleotide backbone or of a polynucleotide side chain may include, or be attached, to a polynucleotide, which polynucleotide may have a sequence capable of non-covalently attaching to another polynucleotide by Watson-Crick base-pair hybridization. For example, a polynucleotide attached to a modified nucleobase may include a sequence of polynucleotides which sequence of polynucleotides is capable of hybridizing with a sequence of nucleotides in another polynucleotide, to form a polynucleotide that is double-stranded over the hybridized sequences. In an example, a polynucleotide side chain may form a non-covalent bond to a modified nucleobase, wherein the modified nucleobase is modified to include a polynucleotide or oligonucleotide, which polynucleotide or oligonucleotide includes a nucleotide sequence complementary to a nucleotide sequence of the polynucleotide side chain, which attached to the modified nucleobase by hybridizing to the complementary sequence.

In an example, nucleotides including such a modification of a nucleobase may be added to a nascent polynucleotide strand. For example, a polymerase may be used to form a polynucleotide backbone, whether based on a template strand for polymerization by a template-dependent polymerase, or polymerization by a template-independent polymerase (e.g., a terminal deoxynucleotidyl transferase). Subsequent to such polynucleotide backbone synthesis, a linking reaction may be performed by attaching a polynucleotide to a modified nucleobase, wherein the polynucleotide includes a chemical moiety capable of attachment to the chemical moiety that is the modification of the modified nucleobase according to a chemical reaction appropriate to forming such an attachment.

In another example, polynucleotides may be attached to nucleotides including such a modification of a nucleobase before such nucleotides are added to a nascent polynucleotide strand. For example, a linking reaction may be performed by attaching a polynucleotide to a modified nucleobase, wherein the polynucleotide includes a chemical moiety capable of attachment to the chemical moiety that is the modification of the modified nucleobase according to a chemical reaction appropriate to forming such an attachment. Then, a polymerase may be used to form a polynucleotide backbone, whether based on a template strand for polymerization by a template-dependent polymerase, or polymerization by a template-independent polymerase (e.g., a terminal deoxynucleotidyl transferase), whereby nucleotides including a polynucleotide attached to a modified nucleobase may be attached to the nascent strand.

Both of the foregoing examples of making a polymer include making a polynucleotide bottlebrush polymer. A polynucleotide backbone includes nucleotides with modified nucleobases, and primary polynucleotide side chains are attached to said nucleobases.

In another example, which may incorporate or combine aspects of any of the foregoing examples, an analogous method may be used to attach one or more secondary nucleotide side chains to modified nucleobases of primary polynucleotide side chains. For example, nucleotides including an aforementioned modification of a nucleobase may be added to a free, 3′ end of a primary polynucleotide side chain, whether based on a template strand for polymerization by a template-dependent polymerase, or polymerization by a template-independent polymerase (e.g., a terminal deoxynucleotidyl transferase). Subsequent to such addition of nucleotides with a modified nucleobase to a primary polynucleotide side chain, a linking reaction may be performed by attaching a polynucleotide to a modified nucleobase of a primary polynucleotide side chain, wherein the polynucleotide to be attached includes a chemical moiety capable of attachment to the chemical moiety that is the modification of the modified nucleobase, according to a chemical reaction appropriate to forming such an attachment.

In another example, which may incorporate or combine aspects of any of the foregoing examples, polynucleotides may be attached to nucleotides including such a modification of a nucleobase before such nucleotides are added to a 3′ end of a primary polynucleotide side chain. For example, a linking reaction may be performed by attaching a polynucleotide to a modified nucleobase, wherein the polynucleotide includes a chemical moiety capable of attachment to the chemical moiety that is the modification of the modified nucleobase according to a chemical reaction appropriate to forming such an attachment. Then, a polymerase may be used to add such a nucleotide to a 3′ end of a primary polynucleotide side chain, whether based on a template strand for polymerization by a template-dependent polymerase, or polymerization by a template-independent polymerase (e.g., a terminal deoxynucleotidyl transferase), whereby nucleotides including a polynucleotide attached to a modified nucleobase may be attached to the primary polynucleotide side chain extended by the polymerase.

In another example, which may incorporate or combine aspect of any of the foregoing examples, a polynucleotide that is attached to a modified nucleotide of a polynucleotide backbone, thereby becoming a primary polynucleotide side chain, may itself include nucleotides with a modified nucleobase. For example, a polynucleotide to be attached to a polynucleotide backbone to become a primary polynucleotide side chain may, before such attaching, include one or more nucleotide with a modification of a nucleobase, such as a chemical moiety for subsequent attachment of a polynucleotide thereto. Said polynucleotide for subsequent attachment thereto may be attachable by inclusion therein of a chemical moiety adapted for attachment to the moiety of the modified nucleobase of a nucleotide of the polynucleotide to be attached to the polynucleotide backbone (thereby becoming a primary polynucleotide side chain) by an appropriate chemistry. As disclosed above for incorporation into a polynucleotide backbone of one or more nucleotide with a modified nucleobase including a chemical moiety for subsequent attachment of a primary polynucleotide side chain, one or more nucleotide with a modified nucleobase including a chemical moiety for subsequent attachment of a secondary polynucleotide side chain may be incorporated into a polynucleotide side chain before it is attached to a polynucleotide backbone to become a primary polynucleotide side chain. Such incorporation my be done using a polymerase, including a template-dependent polymerase or a template-independent polymerase (e.g., a terminal deoxynucleotidyl transferase).

Explicitly, all examples herein for addition, to a polynucleotide backbone, of a nucleotide with a modified nucleobase including a chemical moiety for subsequent attachment to a polynucleotide, are also equally applicable for adding such a nucleotide to a polynucleotide which polynucleotide may be attached to a modified nucleotide of a polynucleotide backbone to become a primary polynucleotide side chain. A modification of a nucleobase of nucleotide of a polynucleotide backbone, or of a nucleobase of a nucleotide for addition to a polynucleotide backbone such as by a polymerase, is referred to generally herein as a primary modification, signifying that it is modification of a nucleotide for attachment to a primary polynucleotide side chain. A modification of a nucleobase of nucleotide of a primary polynucleotide side chain, or of a nucleobase of a nucleotide for addition to a primary polynucleotide side chain such as by a polymerase, is referred to generally herein as a secondary modification, signifying that it is a modification of a nucleotide for attachment to a secondary polynucleotide side chain. A modification of a nucleobase of nucleotide of a secondary polynucleotide side chain, or of a nucleobase of a nucleotide for addition to a secondary polynucleotide side chain such as by a polymerase, is referred to generally herein as a tertiary modification, signifying that it is a modification of a nucleotide for attachment to a tertiary polynucleotide side chain. A modification of a nucleobase of nucleotide of a tertiary polynucleotide side chain, or of a nucleobase of a nucleotide for addition to a tertiary polynucleotide side chain such as by a polymerase, is referred to generally herein as a quaternary modification, signifying that it is a modification of a nucleotide for attachment to a quaternary polynucleotide side chain. Generally speaking, a modification of a nucleobase of nucleotide of a polynucleotide side chain of an n generation, or of a nucleobase of a nucleotide for addition to a polynucleotide side chain of an n generation such as by a polymerase, is referred to generally herein as a (n+1)-ary modification, signifying that it is a modification of a nucleotide for attachment to a polynucleotide side chain of an (n+1) generation.

A polymer as disclosed herein may have a number of primary, or secondary, or tertiary, or quaternary, or n-ary side chain polynucleotides within a range. The range may be from about 1,000 to about 10,000, or from about 5 to about 15, or from about 10 to about 25, or from about 10 to about 100, or from about 15 to about 50, of from about 25 to about 75, or from about 50 to about 150, or from about 100 to about 200, or from about 150 to about 250, or from about 200 to about 300, or from about 250 to about 350, or from about 300 to about 400, or from about 350 to about 450, or from about 400 to about 500, or from about 500 to about 650, or from about 600 to about 750, or from about 700 to about 850, or from about 800 to about 1,000, or from about 900 to about 1,150, or from about 1,000 to about 1,200, or from about 1,100 to about 1,300, or from about 1,200 to about 1,400, or from about 1,300 to about 1,500, or from about 1,400 to about 1,600, or from about 1,500 to about 1,750, or from about 1,700 to about 1,950, or from about 1,900 to about 2,150, or from about 2,100 to about 2,350, or from about 2,300 to about 2,550, or from about 2,500 to about 2,750, or from about 2,700 to about 2,950, or from about 2,900 to about 3,150, or from about 3,100 to about 3,600, or from about 3,500 to about 4,000, or from about 3,900 to about 4,400, or from about 4,300 to about 4,800, or from about 4,700 to about 5,200, or from about 5,100 to about 5,600, or from about 5,500 to about 6,000, or from about 5,750 to about 6,500, or from about 6,250 to about 7,000, or from about 6,750 to about 7,500, or from about 7,250 to about 8,000, or from about 7,750 to about 8,500, or from about 8,250 to about 9,000, or from about 8,750 to about 9,500, or from about 9,250 to about 10,000. The range may be within any sub-range or overlapping range within or between the foregoing examples.

A polynucleotide backbone, or a polynucleotide side chain of a generation of polynucleotide side chains of a polymer as disclosed herein may have a number of side chain polynucleotides of the subsequent generation of polynucleotide side chains attached to modified nucleobases of nucleotides thereof. The number may be within a range, and the range may be from about 1,000 to about 10,000, or from about 5 to about 15, or from about 10 to about 25, or from about 10 to about 100, or from about 15 to about 50, of from about 25 to about 75, or from about 50 to about 150, or from about 100 to about 200, or from about 150 to about 250, or from about 200 to about 300, or from about 250 to about 350, or from about 300 to about 400, or from about 350 to about 450, or from about 400 to about 500, or from about 500 to about 650, or from about 600 to about 750, or from about 700 to about 850, or from about 800 to about 1,000, or from about 900 to about 1,150, or from about 1,000 to about 1,200, or from about 1,100 to about 1,300, or from about 1,200 to about 1,400, or from about 1,300 to about 1,500, or from about 1,400 to about 1,600, or from about 1,500 to about 1,750, or from about 1,700 to about 1,950, or from about 1,900 to about 2,150, or from about 2,100 to about 2,350, or from about 2,300 to about 2,550, or from about 2,500 to about 2,750, or from about 2,700 to about 2,950, or from about 2,900 to about 3,150, or from about 3,100 to about 3,600, or from about 3,500 to about 4,000, or from about 3,900 to about 4,400, or from about 4,300 to about 4,800, or from about 4,700 to about 5,200, or from about 5,100 to about 5,600, or from about 5,500 to about 6,000, or from about 5,750 to about 6,500, or from about 6,250 to about 7,000, or from about 6,750 to about 7,500, or from about 7,250 to about 8,000, or from about 7,750 to about 8,500, or from about 8,250 to about 9,000, or from about 8,750 to about 9,500, or from about 9,250 to about 10,000. The range may be within any sub-range or overlapping range within or between the foregoing examples.

A polynucleotide backbone, or a primary polynucleotide side chain, of a bottlebrush polymer as disclosed herein may include a ratio of nucleotides including primary modifications or secondary modification, respectively, to nucleotides without such modifications. Such a ratio may be from about 100:1, about 100:2, about 100:3, about 100:4, about 100:5, about 100:6, about 100:7, about 100:8, about 100:9, about 100:10, about 100:11, about 100:12, about 100:13, about 100:14, about 100:15, about 100:16, about 100:17, about 100:18, about 100:19, about 100:20, about 100:21, about 100:22, about 100:23, about 100:24, about 100:25, about 100:26, about 100:27, about 100:28, about 100:29, about 100:30, about 100:31, about 100:32, about 100:33, about 100:34, about 100:35, about 100:36, about 100:37, about 100:38, about 100:39, about 100:40, about 100:41, about 100:42, about 100:43, about 100:44, about 100:45, about 100:46, about 100:47, about 100:48, about 100:49, about 100:50, about 100:51, about 100:52, about 100:53, about 100:54, about 100:55, about 100:56, about 100:57, about 100:58, about 100:59, about 100:60, about 100:61, about 100:62, about 100:63, about 100:64, about 100:65, about 100:66, about 100:67, about 100:68, about 100:69, about 100:70, about 100:71, about 100:72, about 100:73, about 100:74, about 100:75, about 100:76, about 100:77, about 100:78, about 100:79, about 100:80, about 100:81, about 100:82, about 100:83, about 100:84, about 100:85, about 100:86, about 100:87, about 100:88, about 100:89, about 100:90, about 100:91, about 100:92, about 100:93, about 100:94, about 100:95, about 100:96, about 100:97, about 100:98, about 100:99, and about 100:100.

Such a ratio may be from about 1:100, about 2:100, about 3:100, about 4:100, about 5:100, about 6:100, about 7:100, about 8:100, about 9:100, about 10:100, about 11:100, about 12:100, about 13:100, about 14:100, about 15:100, about 16:100, about 17:100, about 18:100, about 19:100, about 20:100, about 21:100, about 22:100, about 23:100, about 24:100, about 25:100, about 26:100, about 27:100, about 28:100, about 29:100, about 30:100, about 31:100, about 32:100, about 33:100, about 34:100, about 35:100, about 36:100, about 37:100, about 38:100, about 39:100, about 40:100, about 41:100, about 42:100, about 43:100, about 44:100, about 45:100, about 46:100, about 47:100, about 48:100, about 49:100, about 50:100, about 51:100, about 52:100, about 53:100, about 54:100, about 55:100, about 56:100, about 57:100, about 58:100, about 59:100, about 60:100, about 61:100, about 62:100, about 63:100, about 64:100, about 65:100, about 66:100, about 67:100, about 68:100, about 69:100, about 70:100, about 71:100, about 72:100, about 73:100, about 74:100, about 75:100, about 76:100, about 77:100, about 78:100, about 79:100, about 80:100, about 90:100, about 91:100, about 92:100, about 93:100, about 94:100, about 95:100, about 96:100, about 97:100, and about 98:100, about 99:100.

A primary modification or a secondary modification may include modification of a nucleobase to include a chemical moiety of a pair of chemical moieties the pair being attachable to each other by a suitable chemical reaction, such as a click chemistry reaction. Non-limiting examples of such pairs include: (i) azido/alkynyl; (ii) alkynyl/azido; (iii) thiol/alkynyl; (iv) alkynyl/thiol; (v) alkenyl/thiol; (vi) thiol/alkenyl; (vii) azido/cyclooctynyl; (viii) cyclooctynyl/azido; (ix) nitrone/cyclooctynyl; and (x) cyclooctynyl/nitrone. For example, the modification of the nucleobase may be an azido and the modification of the polynucleotide for attachment thereto may be an alkynyl, or vice versa.

In some examples, the click chemistry reaction includes copper catalyzed azide-alkyne cycloaddition (CuAAC). The covalent linkage may include a triazolyl. The CuAAC may include a Cu(I) stabilizing ligand. The Cu(I) stabilizing ligand may be selected from the group consisting of: 3-[4-({bis[(1-tert-butyl-1H-1,2,3-triazol-4-yl)methyl]amino}methyl)-1H-1,2,3-triazol-1-yl]propanol (BTTP), 3-[4-({bis[(1-tert-butyl-1H-1,2,3-triazol-4-yl)methyl]amino}methyl)-1H-1,2,3-triazol-1-yl]propyl hydrogen sulfate (BTTPS), 2-4-({bis[(1-tert-butyl-1H-1,2,3-triazol-4-yl)methyl]amino}methyl)-1H-1,2,3-triazol-1-yl]ethyl hydrogen sulfate (BTTES), 2-4-{(bis[(1-tert-butyl-1H-1,2,3-triazol-4-yl)methyl]amino)methyl}-1H-1,2,3-triazol-1-yl]-acetic acid (BTTAA), bathophenanthroline disulfonate disodium salt (BPS), N,N,N′,N″,N″-Pentamethyldiethylenetriamine (PMDETA), tris-((1-benzyl-1H-1,2,3-triazol-4-yl)methyl)amine (TBTA), Tris(3-hydroxypropyltriazolylmethyl)amine (THPTA), N^{ε}-((1R,2R)-2-azidocyclopentyloxy)carbonyl)-L-lysine (ACPK), and 4-N,N-dimethyl amino-1,8-naphthalimide (4-DMN).

In some examples, the click chemistry reaction includes strain-promoted azide-alkyne cycloaddition (SPAAC). The covalent linkage may include a cycloocta-triazolyl. In some examples, the click chemistry reaction includes alkyne hydrothiolation. The covalent linkage may include an alkenyl sulfide. In some examples, the click chemistry reaction includes alkene hydrothiolation. The covalent linkage may include an alkyl sulfide. In some examples, the click chemistry reaction includes strain-promoted alkyne-nitrone cycloaddition (SPANC). The covalent linkage may include an octahydrocycloocta-isoxazolyl. The cyclooctynyl may be dibenzylcyclooctyne (DBCO) or a derivative thereof. In some examples, the click chemistry reaction is biocompatible.

A non-exclusive list of complementary binding partners is presented in Table 1:

| | Example moiety/structure on | Example moiety/structure on |
|---|---|---|
| | (a) for forming chemical | (b) for forming chemical |
| | attachment with a | attachment with a |
| | corresponding moiety/structure | corresponding |
| Bonding site | (b) | moiety/structure (a) |
| amine-NHS | amine group, —NH₂ | 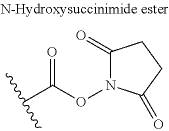 [SMILES: *C(=O)ON1C(=O)CCC1=O] |
| amine-imidoester | amine group, —NH₂ | 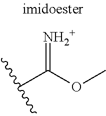 [SMILES: *C(=[NH2+])OC] |
| amine-pentofluorophenyl ester | amine group, —NH₂ | 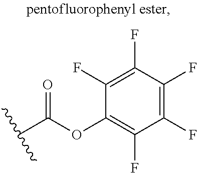 [SMILES: *C(=O)Oc1c(F)c(F)c(F)c(F)c1F] |
| amine-hydroxymethyl phosphine | amine group, —NH₂ | 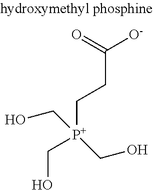 [SMILES: O=C([O-])CC[P+](CO)(CO)CO] |
| amine-carboxylic acid | amine group, —NH₂ | carboxylic acid group, |
| | | —C(=O)OH (e.g., following |
| | | activation of the carboxylic |
| | | acid by a carbodiimide such |
| | | as EDC (1-ethyl-3-(-3- |
| | | dimethylaminopropyl) |
| | | carbodiimide hydrochloride) |
| | | or DCC (N′, N′-dicyclohexyl |
| | | carbodiimide) to allow for |
| | | formation of an amide bond |
| | | of the activated carboxylic |
| | | acid with an amine group) |
| thiol-maleimide | thiol, —SH | 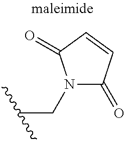 [SMILES: *CN1C(=O)C=CC1=O] |
| thiol-haloacetyl | thiol, —SH | 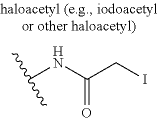 [SMILES: *NC(=O)CI] |
| thiol-pyridyl disulfide | thiol, —SH | 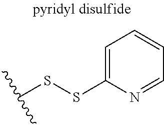 [SMILES: *SSc1ccccn1] |
| thiol-thiosulfonate | thiol, —SH | 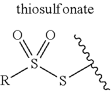 |
| thiol-vinyl sulfone | thiol, —SH | 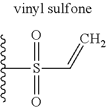 [SMILES: *S(=O)(=O)C=C] |
| aldehyde-hydrazide | aldehyde, —C(═O)H | 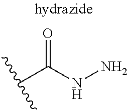 [SMILES: *C(=O)NN] |
| aldehyde-alkoxyamine | aldehyde, —C(═O)H | 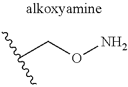 [SMILES: *CON] |
| hydroxy-isocyanate | hydroxyl, —OH | 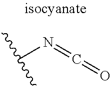 [SMILES: *N=C=O] |
| azide-alkyne | azide, —N₃ | 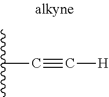 [SMILES: *C#C] |
| azide-phosphine | azide, —N₃ | 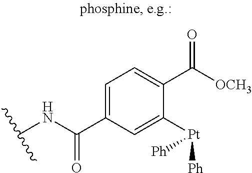 |
| azide-cyclooctyne | azide, —N₃ | 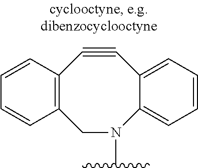 [SMILES: *N1Cc2ccccc2C#Cc2ccccc21] |
| | | 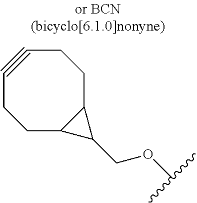 [SMILES: *OCC1C2CCC#CCCC21] |
| azide-norbornene | azide, —N₃ | 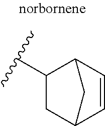 [SMILES: *C1CC2C=CC1C2] |
| transcyclooctene- tetrazine | 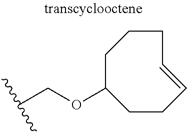 [SMILES: *COC1CC/C=C/CCC1] | 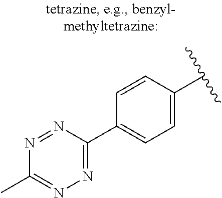 [SMILES: *c1ccc(-c2nnc(C)nn2)cc1] |
| norbornene-tetrazine | 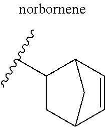 [SMILES: *C1CC2C=CC1C2] | 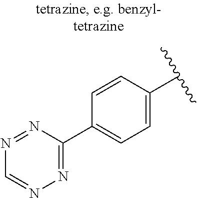 [SMILES: *c1ccc(-c2nncnn2)cc1] |
| oxime | aldehyde or ketone (e.g., amine | alkoxyamine |
| | group or N-terminus of | |
| | polypeptide converted to an | |
| | aldehyde or ketone by | |
| | pyroxidal phosphate) | |
| SpyTag-SpyCatcher | SpyTag: amino acid sequence | SpyCatcher amino acid |
| | AHIVMVDAYKPTK | sequence: |
| | | MKGSSHHHHHHVDIPTT |
| | | ENLYFQGAMVDTLSGLS |
| | | SEQGQSGDMTIEEDSATH |
| | | IKFSKRDEDGKELAGAT |
| | | MELRDSSGKTISTWISDG |
| | | QVKDFYLYPGKYTFVET |
| | | AAPDGYEVATAITFTVNE |
| | | QGQVTVNGKATK |
| SNAP-tag-O⁶- Benzylguanine | SNAP-tag (O-6- methylguanine-DNA methyltransferase) | 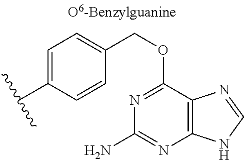 [SMILES: *c1ccc(COc2nc(N)nc3[nH]cnc23)cc1] |
| CLIP-tag-O²- benzylcytosine | CLIP-tag (modified O-6- methylguanine-DNA methyltransferase) | 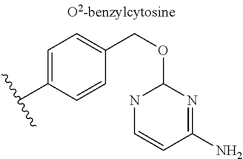 [SMILES: *c1ccc(COC2N=C(N)C=CN2)cc1] |
| Sortase-coupling | -Leu-Pro-X-Thr-Gly | -Gly₍₃₋₅₎ |

Any of the foregoing may be included in or added to a nucleobase to be a primary or secondary modification, as disclosed herein for attachment to a polynucleotide, which polynucleotide may include or be modified to include a complementary moiety or structure of the foregoing pairs for bonding to the primary or secondary modification.

Any of the foregoing may also be included for attaching an amino acid sequence to a modified nucleobase. For example, a modified nucleobase may include a chemical moiety for forming a covalent attachment, such as in an example of a moiety for bonding with another by click chemistry or other covalent attachment chemistry, and an amino acid in an amino acid sequence may include as a modification a chemical moiety for forming at attachment to the chemical moiety of the modified nucleobase. The amino acid in the amino acid sequence may also include a polypeptide for forming a covalent or non-covalent attachment with a polynucleotide side chain. For example, the amino acid may include a polypeptide from among those disclosed as a member of a bonding pair in Table 1. A polynucleotide side chain, in turn, may include a polypeptide or chemical moiety that is a bonding pair complement, as disclosed in Table 1, to the polypeptide included in the amino acid sequence. Or, the amino acid sequence and polynucleotide side chain may include polypeptides capable of forming an avidin-biotin peptide-peptide attachment with each other, or a coiled-coil peptide attachment with each other. Examples of avidin-biotin peptide-peptide attachment and of coiled-coil peptide attachment included in this disclosure are explicitly included as non-limiting examples of non-covalent attachment of a polynucleotide side chain to a modified nucleobase of a nucleotide of a polymer disclosed herein.

Any suitable bioconjugation methods for adding or forming bonds between such pairs of complementary moieties or structures may be used. Modified nucleotides may be commercially available possessing examples of one or the other of examples of such pairs of complementary moieties or structures, and methods for including one or more of such examples of moieties or structures in or attaching or including them to polymer, a nucleotide, or polynucleotide are also known. Also commercially available are bifunctional linker molecules with a moiety or structure from one complementary pair of bonding partners listed in Table 1 at one end and a moiety or structure from another complementary pair of bonding partners listed in Table 1. A moiety or structure of a primary or secondary modification, or a polynucleotide for attachment thereto, may be bound to one end of such a linker, resulting in the initial moiety or structure being effectively replaced with another, i.e., the moiety or structure present on the other end of the bifunctional linker.

For example, a bifunctional linker may have on one end a moiety from among those listed in Table 1, such as an NHS-ester group. At the other end it may have another group, such as an azide group. The ends may be connected to each other by a linker, such as, for example, one or more PEG groups, alkyl chain, combinations thereof in a linking sequence, etc. If a bonding site (such as of primary or secondary modification, or a polynucleotide for attachment thereto) has an amine group for bonding, the NHS-ester end of the bifunctional linker may be bound to the amine group, leaving the free azide end available for bonding to a composition (such as a polynucleotide for attachment to a primary or secondary modification, or a primary or secondary modification) bearing a bonding partner for an azide group (e.g., alkyne, phosphine, cyclooctyne, or norbornene). Or, if a bonding site has a bonding partner for an azide group (e.g., alkyne, phosphine, cyclooctyne, or norbornene), the azide end of the bifunctional linker may be bound to the amine group, leaving the free NHS-ester end available for bonding to a composition bearing an amine group. Many other examples of bifunctional linkers are commercially available including on an end a moiety identified in Table 1 for forming one type of bonding site and on the other end a different moiety identified in Table 1 for forming another type of bonding site.

A linker linking a chemical moiety for forming a chemical attachment may be an aliphatic carbon chain. In some examples, the aliphatic carbon chain may be saturated. In some examples, the aliphatic carbon chain may be unsaturated. In some examples, the aliphatic carbon chain may be substituted. In some examples, the aliphatic carbon chain may be unsubstituted. In some examples, the aliphatic carbon chain may be straight. In some examples, the aliphatic carbon chain may be branched. In some examples, the length of the aliphatic carbon chain may be, or may be about, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values. In some examples, the length of the aliphatic carbon chain may be at least, may be at least about, may be at most, or may be at most about, 2, 3, 4, 5, 6, 7, 8, 9, 10, 20, 30, 40, 50, 60, 70, 80, 90, 100, 200, 300, 400, 500, 600, 700, 800, 900, 1000. The aliphatic carbon chain may include polyethylene glycol (PEG). In some examples, the PEG has a n, the number of the ethylene glycol (—OCH2CH2—) repeating unit, of, or of about, 1, 2, 3, 4, 5, 6, 7, 8, 9, 10, 20, 30, 40, 50, 60, 70, 80, 90, 100, or a number or a range between any two of these values. In some examples, the PEG has a n, the number of ethylene glycol (—OCH2CH2—) repeating unit, of at least, of at least about, or at most, or of at most about, 1, 2, 3, 4, 5, 6, 7, 8, 9, 10, 20, 30, 40, 50, 60, 70, 80, 90, or 100. Linkers may differ from modified nucleobase to modified nucleobase ad from example to example.

A ratio of nucleotides in a polynucleotide having a primary or secondary modification to nucleotides of the polynucleotide not having such modification may be determined during, for example, a method of adding nucleotides having the primary or secondary modification to the polynucleotide. As used herein, a “nucleotide” includes a nitrogen-containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. Nucleotides having a modified base are commercially available wherein a modification includes a moiety for subsequent chemical attachment thereto of a polynucleotide with a complementary chemical moiety. In other examples, a nucleobase may be modified to include a modification (including by attaching a bifunctional linker to a site on a modified nucleobase as described above). A modified base may include a modified adenine, a modified guanine, a modified cytosine, a modified thymine, or a modified uracil. A modified nucleotide may include, as non-limiting examples, 5-(15-Azido-4,7,10,13-tetraoxa-pentademayoyl-aminoallyl)-2′-deoxyuridine-5′-triphosphate (Azide-PEG4-aminoallyl-dUTP), N⁶-(6-Azido)hexyl-3′-deoxyadenosine-5′-triphosphate (N⁶-(6-Azido)hexyl-3′-dATP), 5-(Octa-1,7-diynyl)-2′-deoxycytidine 5′-triphosphate, 5-(Octa-1,7-diynyl)-2′-deoxyuridine 5′-triphosphate, 5-Ethynyl-2′-deoxyuridine 5′-triphosphate, 5-Dibenzylcyclooctyl-PEG4-deoxycytidine-5′-triphosphate, 5-Dibenzylcyclooctyl-PEG4-uridine-5′-triphosphate, 5-trans-Cyclooctene-PEG4-dUTP, 7-Deaza-7-propargylamino-2′-deoxyadenosine-5′-triphosphate or a combination thereof. Other examples of nucleotides including modified nucleobases that may be included in a polymer as disclosed herein are disclosed in Table 12 of U.S. Pat. No. 10,023,626, the entire contents of which is hereby incorporated by reference in its entirety.

Nucleotides bearing a primary or secondary modification may be added to a polynucleotide using a template-dependent polynucleotide (e.g., a DNA Pol I or other suitable template-dependent DNA polymerase). A single-stranded template polynucleotide may be provided including nucleotides including two or more types of nucleobases (from among A, G, T, and C). The template may be contacted with a primer oligonucleotide that is complementary a portion of the template, a template dependent polymerase, and nucleoside triphosphates. Based on Watson-Crick base pairing, the polymerase incorporates nucleoside triphosphates into the 3′ end of the primer, in succession, according to what complementary nucleotide is present on the template. As the polymerase moves along the template starting initially at the 3′ end of the primer and moving towards to 5′ end of the template, incorporating nucleotides into the nascent growing strand whose 5′ end is the primer.

A relative number of nucleotides having modified nucleobases incorporated into the nascent strand may be predetermined by appropriate sequential arrangement of nucleotides in the template and appropriate selection of nucleoside triphosphates bearing a modified nucleobase. As a nonlimiting example, an Azide-PEG4-aminoallyl-dUTP nucleoside triphosphate may be incorporated into a nascent strand of a polynucleotide by a polymerase based on a template. Azide-PEG4-aminoallyl-dUTP is incorporated when a polymerase encounters a T as it moves along a template polynucleotide (i.e., is incorporated by a template-dependent polymerase where an A would normally be incorporated). A sequence of nucleotides in a template polynucleotide may include a T at positions according to where a nucleoside triphosphate is intended to be incorporated into a nascent strand by a template-dependent polymerase, with nucleotides other than T present at other sites of the template polynucleotide. Of the nucleoside triphosphates included in the polymerization reaction, PEG4-aminoallyl-dUTP may be included instead of A, as well as one or more of G, T, and C, according to nucleotides other than T present in the template polynucleotide (being complementary for pairing to C, A, and G in the template polynucleotide, respectively). In other examples, a nucleoside triphosphate with a modified nucleobase other than azide-PEG4-aminoallyl-dUTP may be included, which may added to a nascent polynucleotide strand by a template-dependent polymerase according to a nucleotide (A, G, T, or C) in a polynucleotide template to which it is complementary.

By including a nucleoside triphosphate bearing a primary or secondary modification in a polymerization reaction with a template-dependent polymerase, which nucleoside triphosphate may be incorporated into a nascent strand polymerized by the polymerase according to a template, wherein the template includes nucleotides complementary to the nucleoside triphosphate bearing the modified nucleobase, the number and relative location of nucleotides with modified nucleobases incorporated into the nascent polynucleotide strand may be controlled. For example, a polynucleotide template that includes a plurality of adjacent nucleotides complementary to the nucleoside triphosphate having a modified nucleobase will direct the polymerase to incorporate a plurality of adjacent nucleotides with modified nucleobases in the corresponding region of the nascent strand complementary to the template polynucleotide. In another example, interspersing such nucleotides in the template polynucleotide, with nucleotides therebetween that are not complementary to the nucleoside triphosphates bearing the modified nucleobase, and including additional nucleoside triphosphates complementary to such additional nucleotides in the polymerase reaction, will direct the polymerase to intersperse nucleotides with the modified nucleobase in the nascent polynucleotide strand complementary to the template polynucleotide accordingly. Any desired pattern or concentration of modified nucleotides in a polynucleotide backbone or primary polynucleotide side chain may thereby be included.

In another example, nucleotides including a modified nucleobase may be incorporated into a single-stranded DNA (ssDNA) polynucleotide backbone or primary polynucleotide side chain (or polynucleotide for attachment to a polynucleotide backbone as a primary polynucleotide side chain) using a template-independent polymerase, such as Terminal deoxynucleotidyl Transferase (TdT). A TdT may randomly incorporate nucleotides carrying modifications (such as azide groups on the bases) into a ssDNA polynucleotide strand. However, commercially available TdTs do not readily incorporate multiple serial base modified nucleotides, presumably due to steric clashes.

Examples of recombinant TdTs disclosed herein are thermostable and are better (e.g., much better) than commercially available TdTs, such as TdT from New England Biolabs®, Inc. (NEB; Ipswich, Mass.), at incorporating base modified nucleotides, such as a nucleotide with a PEG chain conjugated to the base (referred to herein as a PEG-nucleotide). For example, NEB TdT would stop after incorporating 1-2 PEG-nucleotides, and a recombinant TdT as disclosed herein may incorporate multiple PEG-nucleotides, including in series.

Any recombinant TdT disclosed herein therefore may be an excellent catalyst for the generation of ssDNA carrying various types of base modified nucleotides for different purposes, including generation of a brush polymer polynucleotide as disclosed herein. A TdT may be contacted with a ssDNA and nucleoside triphosphates and serially incorporate the latter into the ssDNA, in a random order. By including nucleoside triphosphates including modified nucleobases in the reaction, such modified nucleobases may be included in the resulting polynucleotides strand, such as a primary or secondary modification of a polynucleotide backbone or a primary polynucleotide side chain, respectively. In an example, such a polymerase reaction may take place in the presence of no nucleoside triphosphates other than those including a modified nucleobase, resulting in a stretch of modified nucleotides serially incorporated into the ssDNA. In another example, nucleoside triphosphates with and nucleoside triphosphates without a modified nucleobase may both be included in such a polymerase reaction, and a TdT would randomly incorporate them into the ssDNA. In an example, a concentration or relative ratio of such types of nucleoside triphosphates may be included in the reaction, resulting of a relative ratio of nucleotides with and without a primary (or secondary) modification to nucleotides without a modification in the resulting strand, with higher concentrations of the former in the reaction driving a higher ration of modified to unmodified nucleotides in the resulting ssDNA strand and vice versa.

As explained above, in an example, a polynucleotide backbone or a primary polynucleotide side chain may be created by incorporating therein nucleotides with a modified nucleobase wherein the modification includes a chemical moiety that allows subsequent attachment thereto of another polynucleotide including a complementary chemical moiety (with complementary chemical moieties including, for example, pairs of click chemistry binding moieties or other binding pairs as in Table 1). Primary polynucleotide side chains or secondary polynucleotide side chains bearing an appropriate chemical moiety may then be attached to the modified nucleotides of the polynucleotide backbone or a primary polynucleotide side chain. As also explained above, in another example, a polynucleotide backbone or a primary polynucleotide side chain may be created by incorporating therein nucleotides with a nucleobase to which a polynucleotide is already attached, such that a primary polynucleotide side chain or secondary polynucleotide side chain may be added upon addition of the nucleotide with the so-modified nucleobase to the ssDNA polynucleotide backbone or primary polynucleotide side chain. As disclosed herein, a polymerase, as disclosed above, may be used for such incorporations of a nucleotide with a modified nucleobase.

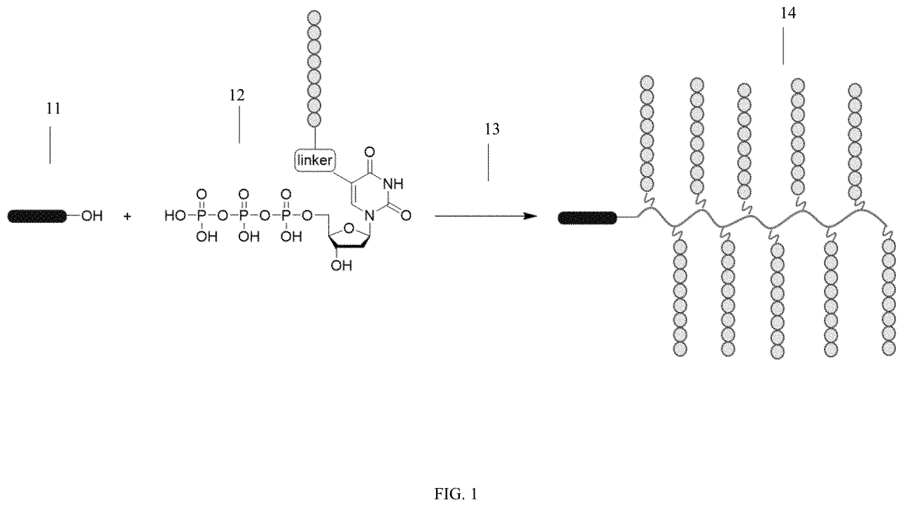

FIG. 1 is an illustration of an example in accordance with the present disclose. A single stranded polynucleotide primer 11 with a free 3′ hydroxyl group is shown, combined with a modified deoxyuridine triphosphate (mod-dUTP) 12 as an example of a nucleoside triphosphate with a modified nucleobase. A polynucleotide is shown attached to the nucleobase of the mod-dUTP. A polymerization reaction 13, catalyzed by a polymerase (not shown) results in addition of multiple mod-dUTP with polynucleotides attached thereto in an example of a polynucleotide backbone (extending from the primer) of a bottlebrush polymer polynucleotide 14, with multiple primary polynucleotide side chains extending therefrom where a mod-dUTP was attached. Not shown, but included within the present disclosure, is inclusion of non-modified nucleotides in the polynucleotide backbone. In this and any following example, a chemical moiety for forming another chemical attachment may be included at an end of the primer opposite to the end to which nucleoside triphosphates with modified nucleobases are added.

A chemical moiety included on a primer preferably is not capable of forming a chemical attachment with a primary modification or secondary modification of the polynucleotide backbone or primary polynucleotide side chin so as to prevent inter-molecular bond formation between the two where disfavored. Similarly, a chemical moiety included in a molecule for forming a chemical bond with the chemical moiety of the primer does not form a chemical bond with a primary modification of a polynucleotide backbone, or secondary modification of a primary polynucleotide side chain, of a bottlebrush polymer polynucleotide. And a chemical moiety included in a molecule for forming a chemical bond with the chemical moiety of a primary modification of a polynucleotide backbone, or secondary modification of a primary polynucleotide side chain, of a bottlebrush polymer polynucleotide does not form a chemical bond with the primer. In that sense, primary and secondary modifications and chemical moieties included on molecules for attachment thereto are said to be orthogonal chemistries to a chemical moieties of a primer and of molecules for forming chemical attachment thereto.

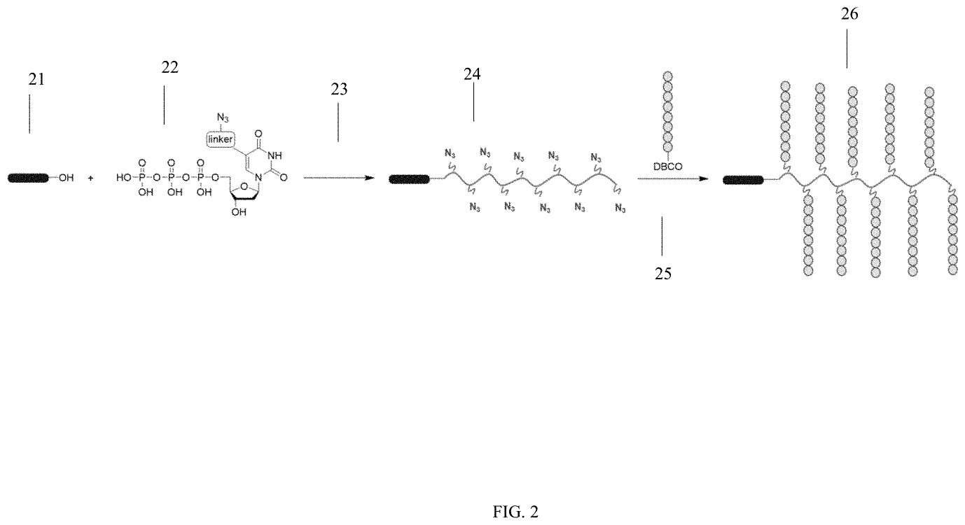

Another example in accordance with the present disclosure is illustrated in FIG. 2. A single stranded polynucleotide primer 21 with a free 3′ hydroxyl group is shown, combined with a modified deoxyuridine triphosphate (mod-dUTP) 22 as an example of a nucleoside triphosphate with a modified nucleobase. In this non-limiting example, an azide (N₃) polynucleotide is shown attached to the nucleobase of the mod-dUTP, though other examples, such as for use in click chemistry reactions or disclosed in Table 1, may be used. A polymerization reaction 23, catalyzed by a polymerase (not shown) results in addition of multiple mod-dUTP with polynucleotides attached thereto in an example of a polynucleotide backbone 24 extending from the primer, with multiple primary modification sites included therein where a mod-dUTP was attached. Not shown, but included within the present disclosure, is inclusion of non-modified nucleotides in the polynucleotide backbone. Following incorporation of nucleotides having primary modifications into the polynucleotide backbone, the polynucleotide with primary modification sites 24 is contacted with polynucleotides having chemical moieties for chemical attachment with the primary modifications 25. In this non-limiting example, the chemical moiety is a cyclooctyne (dibenzocyclooctyne, or DBCO), for forming azide-cyclooctyne bonds to connect primary polynucleotide side chains to the primary modification sites of the polynucleotide backbone by an appropriate click chemistry reaction and form a bottlebrush polymer polynucleotide 26. In other examples, the polynucleotide for attachment to a primary modification site may include any chemical moiety appropriate for attaching to the chemical moiety of the primary modification site, including use of such pairs as in Table 1.

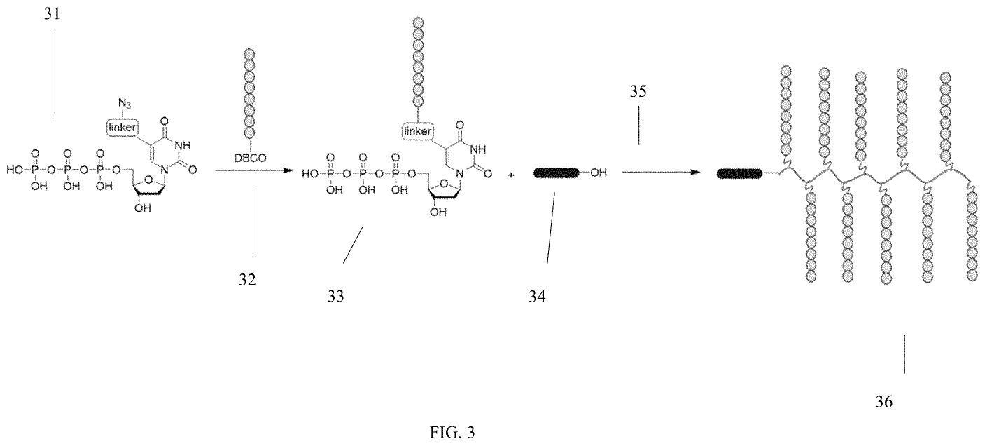

Another example in accordance with the present disclosure is illustrated in FIG. 3. A mod-dUTP having an azide group attached to its modified nucleobase 31 is shown in a chemical reaction with a polynucleotide having a DBCO moiety in a chemical reaction 32 by which a chemical attachment is formed between the mod-dUTP and the polynucleotide. A modified nucleoside triphosphate other than a modified dUTP may also be used, as in all of the examples herein. Here, formation of an azide-cyclooctyne bond is shown as an example of attachment of a polynucleotide to a mod-dUTP but, as above, other pairs of complementary chemical moieties appropriate for attachment to each other may be included on the mod-dUTP and polynucleotide for attachment therebetween. The linking reaction 32 yields a mod-dUTP including a polynucleotide attached to the modified nucleobase thereof. Similar to the example illustrated in FIG. 1, such mod-dUTP 33 may be combined with a primer 34 in the presence of a polymerase (not shown) in a polymerase reaction 35 to form a bottlebrush polymer polynucleotide.

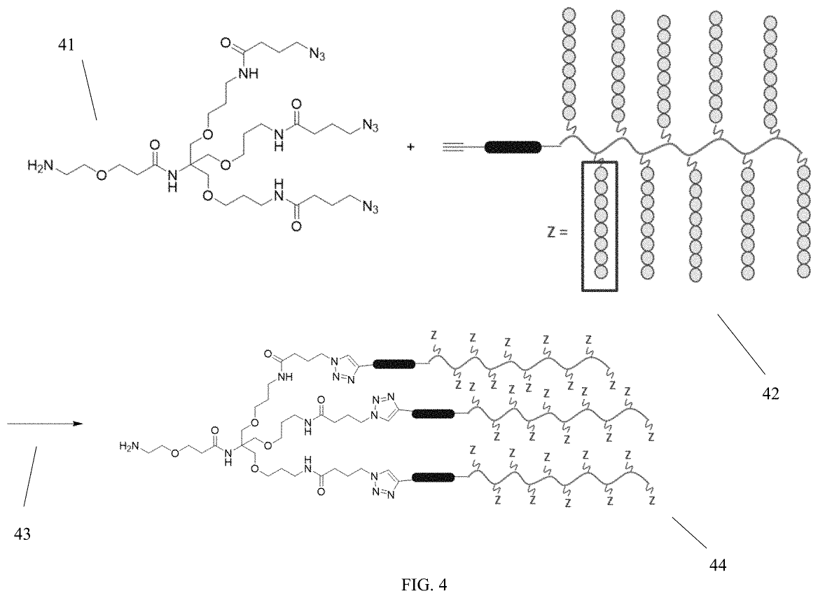

Another example in accordance with the present disclosure is illustrated in FIG. 4. FIG. 4 illustrates an example for conjoining multiple bottlebrush polymer polynucleotides together via a multi-armed scaffold. A non-limiting example of a multi-armed scaffold 41 branches into three ends having a chemical moiety appropriate for attaching to a complementary chemical moiety by an appropriate linking reaction. In this non-limiting example, three branches of a multi-armed scaffold terminate in azide groups, though other examples may be included. A bottlebrush polymer polynucleotide 42 may have a chemical moiety appropriate for attaching to a complementary chemical moiety by an appropriate linking reaction. In this non-limiting example, the primer of the bottlebrush polymer polynucleotide includes a alkyne group. When the branched scaffold 41 and bottlebrush polymer polynucleotide 42 are combined in a linking reaction appropriate for forming a chemical attachment between their respective chemical moieties 43, multiple bottlebrush polymer polynucleotides may be conjoined as shown in the nonlimiting example illustrated at 44. Primary polynucleotide side chains in 44 are represented by Z, as shown in the inset at 42. In this example, the multi-armed scaffold 41 includes a chemical moiety (in this non-limiting example an —NH₂) that could form a chemical attachment to a chemical moiety of another molecule having a chemical moiety complementary to that of the primer (such as, for this non-limiting example, an N-Hydroxysuccinimide ester (NHS), for formation of an amine-NHS bond). Other examples of complementary moieties, such as pairs shown in Table 1, could be used in this example as well for permitting attachment of such end of the multi-arm scaffold to another molecule by the appropriate chemistry.

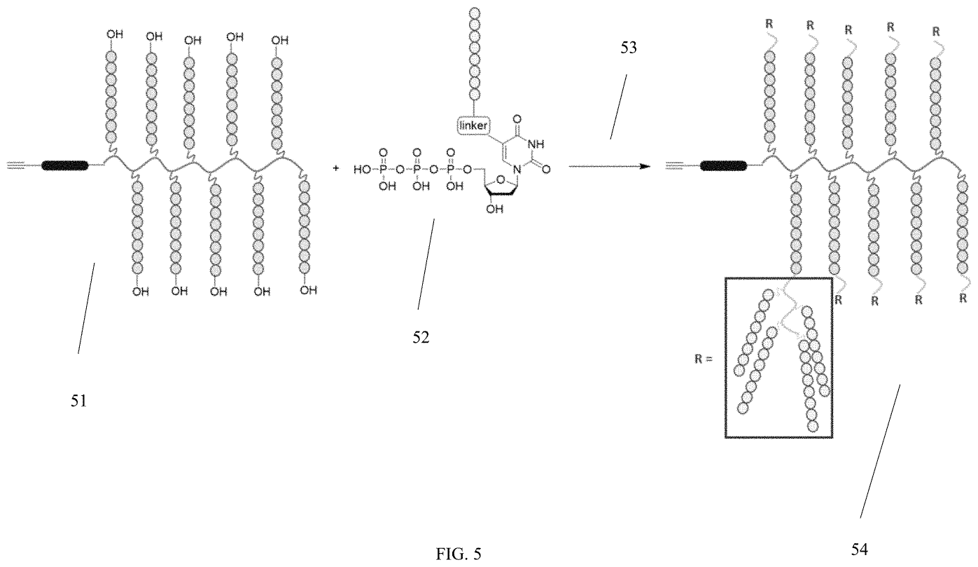

Another example in accordance with the present disclosure is illustrated in FIG. 5. In this example, a bottlebrush polymer polynucleotide 51 includes, on its primer, a chemical moiety for attachment to another molecule (in this non-limiting example, an alkyne group, though other examples of complementary moieties, such as pairs shown in Table 1, could be used in this example as well for permitting attachment of such end of the primer of a bottlebrush polymer polynucleotide to another molecule by the appropriate chemistry). Shown on the primary polynucleotide side chains of the bottlebrush polymer polynucleotide 51 are 3′ hydroxyl groups (—OH). A nucleoside triphosphate having a modified nucleobase is shown 52 wherein the modification is a polynucleotide connected to the nucleoside triphosphate via a linker. A polymerization reaction 53, catalyzed by a polymerase (not shown) adds modified nucleotides to the 3′ ends of the primary polynucleotide side chains. The polynucleotides that are modifications of the nucleobases of the modified polynucleotide triphosphates 52 become secondary polynucleotide side chains of the bottlebrush polymer polynucleotide 54 represented by inset R. In this example, a mod-dUTP is shown as the nucleoside triphosphate 52, though as in the other examples disclosed herein nucleoside triphosphates with other modified nucleobases may be included instead.

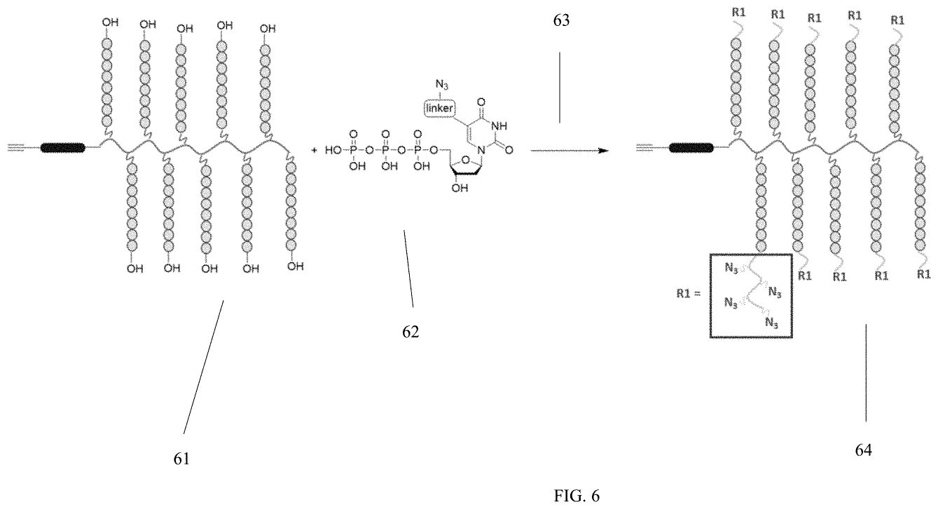

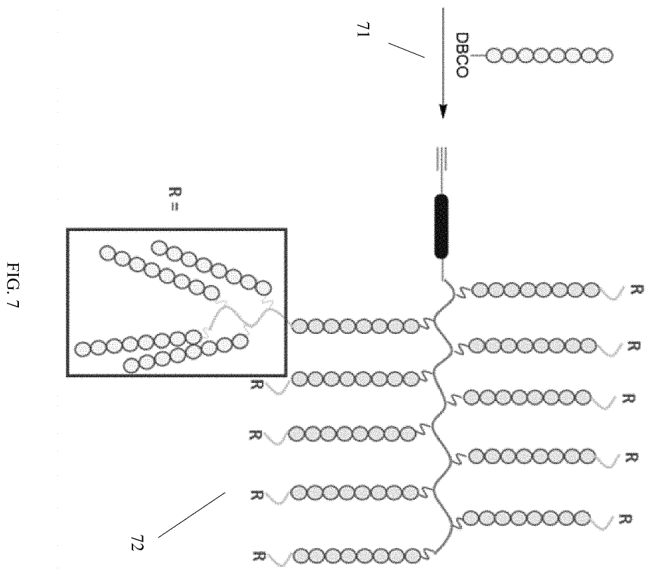

Another example in accordance with the present disclosure is illustrated in FIGS. 6 and 7. In this example, in FIG. 6 a bottlebrush polymer polynucleotide 61 includes, on its primer, a chemical moiety for attachment to another molecule (in this non-limiting example, an alkyne group, though other examples of complementary moieties, such as pairs shown in Table 1, could be used in this example as well for permitting attachment of such end of the primer of a bottlebrush polymer polynucleotide to another molecule by the appropriate chemistry). Shown on the primary polynucleotide side chains of the bottlebrush polymer polynucleotide 51 are 3′ hydroxyl groups (—OH). A nucleoside triphosphate having a modified nucleobase is shown 62 wherein the modification is a chemical moiety for forming a chemical attachment. In this non-limiting example, an azide group is shown as such chemical moiety, although any other chemical moiety appropriate for forming a chemical attachment with molecule including a chemical moiety complementary thereto may be used, such as any of the example from a pair of complementary moieties as shown in Table 1. A polymerization reaction 63, catalyzed by a polymerase (not shown) adds modified nucleotides to the 3′ ends of the primary polynucleotide side chains. The chemical moieties that are modifications of the nucleobases of the modified polynucleotide triphosphates 62 become secondary modifications of the primary polynucleotide side chains of the bottlebrush polymer polynucleotide 64 represented by inset R1

Continuing to FIG. 7, a polynucleotide including a chemical moiety appropriate for forming a chemical attachment with the secondary modification sites is added under conditions for forming a chemical attachment therebetween 71. In this non-limiting example, the polynucleotide includes a DBCO chemical moiety for formation of an azide-cyclooctyne chemical attachment with the secondary modification sites in this example, though other pairs of such chemical moieties may be used as stated above for other example (see e.g. Table 1). Attachment of polynucleotides to the secondary modification sites of the primary polynucleotide side chains results in secondary polynucleotide side chains as shown in the inset R of bottlebrush polymer polynucleotide 72.

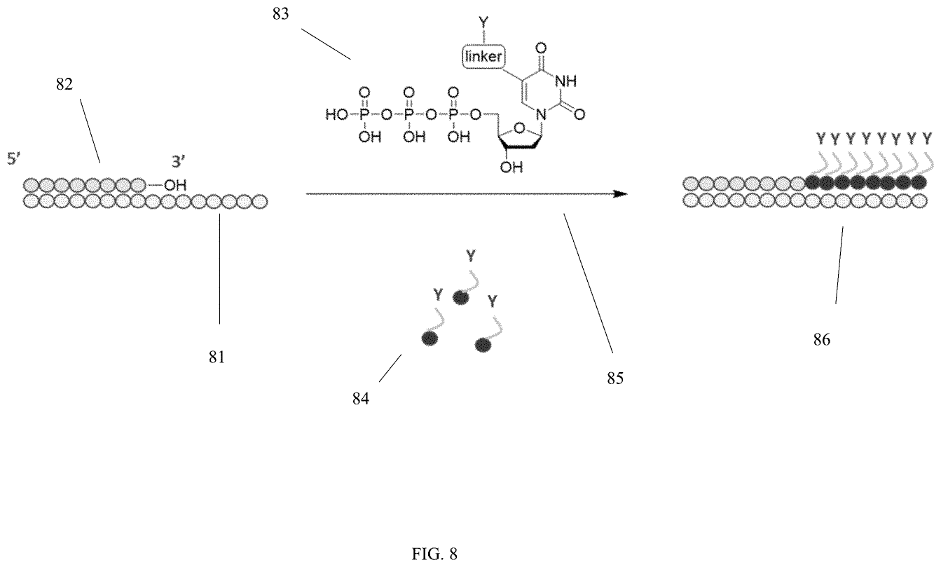

Another example in accordance with the present disclosure is illustrated in FIG. 8. A primer 82 is shown hybridized to a template polynucleotide 81, with the 3′ hydroxyl group of the template polynucleotide shown. Also shown is a nucleoside triphosphate including a modified nucleobase 83. In this non-limiting example mod-dUTP is shown, though other nucleoside triphosphates could be included, with different modified nucleobases. The modification is shown as Y attached to the nucleobase by a linker. In this example, Y may be a chemical moiety for forming a chemical attachment (such as a chemical moiety for a primary modification of a polynucleotide backbone, or a secondary modification for a primary polynucleotide side chain). In a polymerase reaction 85 with a template-dependent polymerase (not shown), multiple copies of the nucleoside triphosphate including the Y-modified nucleobase 84 are attached to the primer 82 extended by the polymerase. The nucleoside triphosphates including the Y-modified nucleobase are complementary to the free, unhybridized nucleotides of the template polynucleotide 81. The polymerase reaction 85 adds nucleotides including the Y-modified nucleobase to the primer as shown at 86. The extended primer and the template polynucleotide may then be dehybridized from each other, though in another example they may stay hybridized to each other included in, or during the formation of, a bottlebrush polymer polynucleotide. Where Y are polynucleotides attached to the modified nucleotides, the structure formed is a bottlebrush polymer polynucleotide. Where Y represents a chemical moiety for chemical attachment to a polynucleotide, Y represents a primary modification or a secondary modification for subsequent attachment to a polynucleotide including a complementary chemical moiety, as explained above.

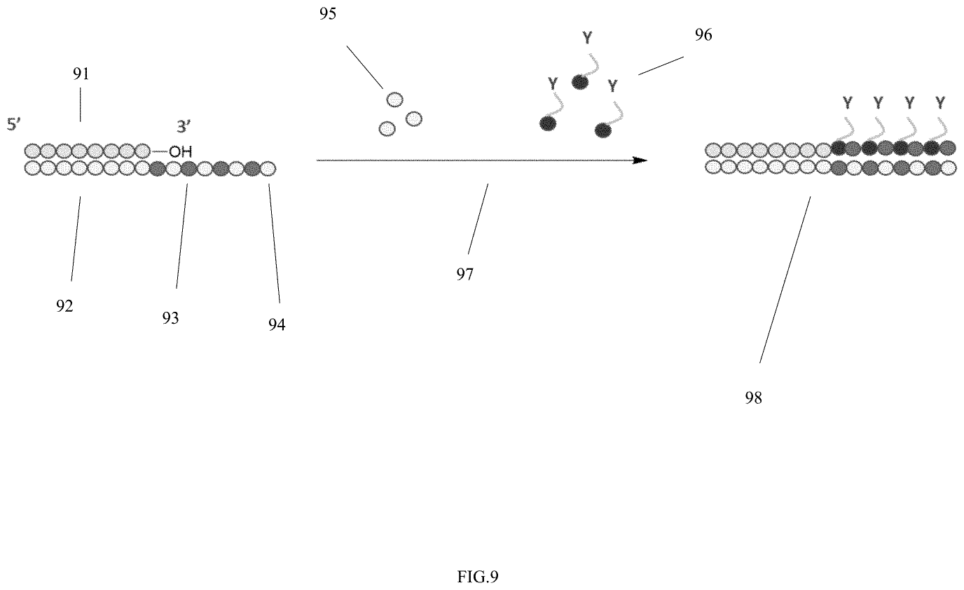

Another example in accordance with the present disclosure is illustrated in FIG. 9. A primer 91 is shown hybridized to a template polynucleotide 92, with the 3′ hydroxyl group of the template polynucleotide shown. Also shown is a nucleoside triphosphate including a modified nucleobase 96, with the modification represented by Y. In this example, Y may be a chemical moiety for forming a chemical attachment (such as a chemical moiety for a primary modification of a polynucleotide backbone, or a secondary modification for a primary polynucleotide side chain). Also shown are nucleoside triphosphates without a modified nucleobase 95. The template includes nucleotides 93 that are complementary to nucleoside triphosphates with a modified nucleobase 96, and nucleotides 94 that are complementary to nucleoside triphosphates that do not include a modified nucleobase 95. In this example, nucleoside triphosphates with a modified nucleobase 96 are not complementary to nucleotides 94 that are complementary to nucleoside triphosphates that do not include a modified nucleobase 95, and nucleoside triphosphates not having a modified nucleobase 95 are not complementary to nucleotides 93 that are complementary to nucleoside triphosphates having a modified nucleobase 96.

In a polymerase reaction 97 with a template-dependent polymerase (not shown), multiple copies of the nucleoside triphosphate including the Y-modified nucleobase 96 and of nucleoside triphosphates not including a modified nucleobase 95 are added to the primer extended by the polymerase. According to the sequence in the template polynucleotide 92 of nucleotides 93 complementary to nucleoside triphosphates including a modified nucleobase Y 96 and nucleotides 94 complementary to nucleoside triphosphates that do not have a modified nucleobase 95, the primer extended by the template-dependent polymerase includes a sequence of nucleotides including a modified nucleobase Y and nucleotides not including a modified nucleobase.

In an example, nucleoside triphosphates that do not have a modified nucleobase 95 may represent multiple types of nucleoside triphosphate not complementary to a nucleotide 93 of the template polynucleotide 92 that is complementary to a nucleoside triphosphate having a modified nucleobase 96. In an example, nucleoside triphosphates having a modified nucleobase 96 may represent multiple types of nucleoside triphosphate not complementary to a nucleotide 94 of the template polynucleotide 92 that is complementary to a nucleoside triphosphate not having a modified nucleobase 95. That is, in any example disclosed herein, nucleotides having more than one type of modified nucleobase may be included in a bottlebrush polymer polynucleotide (e.g., through inclusion of different triphosphate polynucleotides with a modified nucleobase into a ssDNA of a polynucleotide backbone, or a primary or secondary polynucleotide side chain, or any combination of the foregoing).

Examples of nucleoside triphosphates may include a deoxyribose adenine triphosphate, a deoxyribose guanine triphosphate, a deoxyribose cytosine triphosphate, a deoxyribose thymine triphosphate, a deoxyribose uracil triphosphate, or a combination of two or more of the foregoing.

The extended primer and the template polynucleotide may then be dehybridized from each other, though in another example they may stay hybridized to each other included in, or during the formation of, a bottlebrush polymer polynucleotide. Where Y are polynucleotides attached to the modified nucleotides, the structure formed is a bottlebrush polymer polynucleotide. Where Y represents a chemical moiety for chemical attachment to a polynucleotide, Y represents a primary modification or a secondary modification for subsequent attachment to a polynucleotide including a complementary chemical moiety, as explained above.

Examples illustrated in FIGS. 8 and 9 include extension of a strand of DNA by template-dependent extension by a polymerase. Here, a single strand of DNA is extended by the polymerase, and herein extending a ssDNA includes such examples, notwithstanding hybridization of the strand being extended to its template such as during the extending.

Any of a variety of polymerases may be used in a method or composition set forth herein including, for example, protein-based enzymes isolated from biological systems and functional variants thereof. Reference to a particular polymerase, such as those exemplified below, will be understood to include functional variants thereof unless indicated otherwise. A particularly useful function of a polymerase is to catalyze the polymerization of a nucleic acid strand using an existing nucleic acid as a template. Other functions that are useful are described elsewhere herein. Examples of useful polymerases include DNA polymerases. Example DNA polymerases include those that have been classified by structural homology into families identified as A, B, C, D, X, Y, and RT. DNA Polymerases in Family A include, for example, T7 DNA polymerase, eukaryotic mitochondrial DNA Polymerase gamma, E. coli DNA Pol I (including Klenow fragment), Thermus aquaticus Pol I, and Bacillus stearothermophilus Pol I. DNA Polymerases in Family B include, for example, eukaryotic DNA polymerases a, 6, and E; DNA polymerase C; T4 DNA polymerase, Phi29 DNA polymerase, 9°N™, and RB69 bacteriophage DNA polymerase. Family C includes, for example, the E. coli DNA Polymerase III alpha subunit. Family D includes, for example, polymerases derived from the Euryarchaeota subdomain of Archaea. DNA Polymerases in Family X include, for example, eukaryotic polymerases Pol beta, Pol sigma, Pol lamda, and Pol mu, and S. cerevisiae Pol4. DNA Polymerases in Family Y include, for example, Pol eta, Pol iota, Pol kappa, E. coli Pol IV (DINB) and E. coli Pol V (UmuD'2C). The RT (reverse transcriptase) family of DNA polymerases includes, for example, retrovirus reverse transcriptases and eukaryotic telomerases. Other polymerases, disclosed in U.S Pat. No. 8,460,910, which is incorporated herein in its entirety, are also included among polymerases as referred to herein, as are any other functional polymerases including those having sequences modified by comparison to any of the above mentioned polymerase enzymes, which are provided merely as a listing of non-limiting examples. Any recombinant or other TdT disclosed herein may be used as a catalyst for the generation of ssDNA for use in a bottlebrush polymer polynucleotide as disclosed herein

The length of a polynucleotide backbone or primary or secondary polynucleotide side chain may be different in different examples. In some examples, the length of such a polynucleotide may be or may be about, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, or a number or a range between any two of these values, nucleotides. In some examples, such a length may be at least, may be at least about, may be at most, or may be at most about, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, or 10000000, nucleotides.

A temperature at which a ssDNA is contacted with a polymerase and a nucleoside triphosphate including a modified nucleobase for addition thereof to the ssDNA as part of or in relation to a polymerase reaction may be or may be about, −90° C., −89° C., −88° C., −87° C., −86° C., −85° C., −84° C., −83° C., −82° C., −81° C., −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87 ° C., 88° C., 89° C., 90° C., or a number or a range between any two of these values. In some examples, the temperature may be at least, may be at least about, may be at most, or may be at most about, −90° C., −89° C., −88° C., −87° C., −86° C., −85° C., −84° C., −83° C., −82° C., −81° C., −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C. For example, the temperature may be about 20° C. to about 65° C. For example, the temperature may be less than 0° C. For example, the temperature may be about −4° C. to about −20° C.

A dimension (e.g., a diameter) of the polymer including a bottlebrush polymer polynucleotide as disclosed herein may be different in different examples. In some examples, the dimension of the polymer may be, or may be about, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1000 nm, or a number or a range between any two of these values. In some examples, the dimension of the polymer may be at least, may be at least about, i may be s at most, or may be at most about, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm.

In some examples, a recombinant TdT may include an amino acid sequence that is at least 85%, or at least 90% identical, or at least 95% identical, or at least 99% identical, or 100% identical to SEQ ID NO: 1:

MGSSHHHHHHGSGLVPRGSASMSDSEVNQEAKPEVKPEVKPETHINLKVSDGSSEIF FKIKKTTPLRRLMEAFAKRQGKEMDSLRFLYDGIRIQADQTPEDLDMEDNDIIEAHRE QIGGELMRTDYSATPNPGFQKTPPLAVKKISQYACQRKTTLNNYNHIFTDAFEILAEN SVFNGNEVSYVTFMRAASVLKSLPFTIISMKDTEGIPCLGDKVKCIIEEIIEYGESSEVK AVLNDERYQSFKLFTSVFGVGLKTSEKWFRMGFRSLSEIMSDKTLKLTKKQKAGFLY YEDLVSCVTRAEAEAVGVLVKEAVWAFLPDAFVTMTGGFRRGKKIGHDVDFLITSP GSAEDEEQLLPKVINLWEKKGLLLYYDLVESTFEKFKLPSRQVDTLDHFQKCFLILKL PHQRVDSSKSNQQEGKTWKAIRVDLVMCPYENRAFALLGWTGSRQFERDIRRYATH ERKMMLDNHALYDKTKRVFLKAESEEEIFAHLGLDYIEPWERNA.

The recombinant TdT may be thermally stable. The recombinant TdT may be stable at different temperatures in different examples. In some examples, the recombinant TdT may be stable at a temperature of, or of about, 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., or higher. For example, the recombinant TdT may be stable at a temperature of 47° C. or higher. The recombinant TdT may be stable at a temperature of 50° C. or higher. The recombinant TdT may be stable at a temperature of 55° C. or higher. The recombinant TdT may be stable at a temperature of 58° C. or higher. The recombinant TdT may be stable at a temperature of at least, of at least about, of at most, or of at most about, 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., or a number or a range between any two of these values.

### Non-Limiting Examples

The following examples are intended to illustrate particular examples of the present disclosure, but are by no means intended to limit the scope thereof

### Example 1: Template-Dependent Polymerase Incorporation

The incorporation of a base-modified nucleotide (5-(15-Azido-4,7,10,13-tetraoxa-pentademayoyl-aminoallyl)-2′-deoxyuridine-5′-triphosphate (Azide-PEG4-aminoallyl-dUTP; also referred to herein as Az-dU) used as a precursor for preparing an oligonucleotide-linked nucleotide, referred to here as, for example, o-dTTPs) and an o-dTTP (wherein o=an oligonucleotide of 32 A residues) with an SBS polymerase was evaluated in-solution.

Reaction conditions were as follows:

2 μl buffer

0.4 μlMg

2 μl primer-template mix (4 pmol)

2 μl nucleotide (60 pmol)

1 μl Pol (final conc. 0.12 mg/ml)

Templates were 1T (TCTAAGGGTCTGAGGCTCGTCCTGAAT, using the following 1T primer: ATTCAGGACGAGCCTCAGACCCT), 2T (TCTAAGGGTCTGAGGCTCGTCCTGAAT, using the following 2T primer: ATTCAGGACGAGCCTCAGACCC), and 5T (AAAAAGGGTCTGAGGCTCGTCCTGAAT, using the following 5T primer: ATTCAGGACGAGCCTCAGACCC).

dTTP nucleoside triphosphate with a modified nucleobase was Az-dU

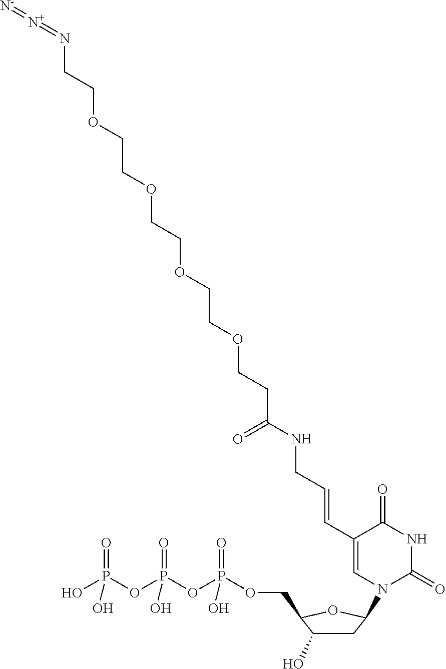 [SMILES: [N-]=[N+]=NCCOCCOCCOCCOCCC(=O)NC/C=C/c1cn([C@H]2C[C@H](O)[C@@H](COP(=O)(O)OP(=O)(O)OP(=O)(O)O)O2)c(=O)[nH]c1=O]

wherein the azide moiety was for binding to a DBCO-attached oligonucleotide of 32 A residues, and o-dTTP was Az-dU with a 32-A oligonucleotide attached via an azide-cyclooctyne chemical attachment.

Up to 5 sequential incorporations of a dTTP or an o-dTTP occurred for a template with 5 A residues. In a separate experiment, up to 10 sequential incorporations of an o-dTTP occurred for a template with 10 A residues (10T: AAAAAAAAAAGGGTCTGAGGCTCGTCCTGAAT).

### Example 2: Template In-Dependent Polymerase Incorporation

TdT polymerases used for template-independent incorporation included NEB TdT and a recombinant TdT having an amino acid sequence of SEQ ID NO:1 (TdT3-2). The NEB TdT was unable to incorporate Az-dU but TdT3-2 incorporated Az-dU into the ssDNA to produce an extended polynucleotide product. In the case of 32A o-NTP , both TdT enzymes performed similarly and incorporated the modified nucleotide into the ssDNA.

Although preferred examples have been depicted and described in detail herein, it will be apparent to those skilled in the relevant art that various modifications, additions, substitutions, and the like may be made without departing from the spirit of the present disclosure and these are therefore considered to be within the scope of the present disclosure as defined in the claims that follow.

1. A method, comprising
extending a ssDNA by sequentially adding a plurality of modified nucleoside triphosphates to the ssDNA, wherein the base of the modified nucleoside triphosphates comprises a primary modification and the primary modification is selected from
(i) a primary polynucleotide attached to the base of the modified nucleoside triphosphate, and
(ii) a site on the base for attachment of a primary polynucleotide to the base, further comprising attaching a primary polynucleotide to the base after the polymerizing.

2. The method of claim 1, wherein the polymerase is a terminal deoxynucleotidyl transferase (TdT).

3. The method of claim 2, wherein the TdT comprises an amino acid sequence that is at least 80% identical to SEQ ID NO: 1.

4. The method of claim 2, wherein the TdT comprises an amino acid sequence of SEQ ID NO: 1.

5. The method of claim 1, wherein a ratio of (a) a number of nucleoside triphosphates comprising the primary modification added to the ssDNA to (b) a number of nucleoside triphosphates not comprising a primary modification added to the ssDNA is from 1:100 to 100:1.

6. The method of claim 1, wherein the polymerase is a template-dependent polymerase.

7. The method of claim 6, wherein a template for the polymerase comprises a ratio of (a) a number of nucleotides complementary to nucleoside triphosphates comprising primary modifications to (b) a number of nucleotides complementary to nucleoside triphosphates not comprising primary modifications, and the ratio is from 1:100 to 100:1.

8. The method of claim 1, wherein the primary polynucleotides further comprise one or more nucleotide comprising a secondary modification, wherein the secondary modification comprises a site on a base for attachment of a secondary polynucleotide side chain.

9. The method of claim 8, wherein the secondary modification comprises a site on a base for covalent attachment of a secondary polynucleotide side chain.

10. The method of claim 8, further comprising attaching a secondary polynucleotide to one or more nucleotide of the primary polynucleotides comprising a secondary modification.

11. The method of claim 10, comprising covalently attaching a secondary polynucleotide to one or more nucleotide of the primary polynucleotides comprising a secondary modification.

12. The method of claim 1, wherein the primary modification comprises a primary polynucleotide covalently attached to the base of the modified nucleoside triphosphate.

13. The method of claim 12, wherein the primary polynucleotide is attached to the base of the modified nucleoside triphosphate by a covalent bond selected from an amine-NHS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

14. The method of claim 1, wherein the primary modification comprises a site on the base for attachment of a primary polynucleotide to the base, wherein the site on the base for attachment of a primary nucleotide to the base is for covalent attachment.

15. The method of claim 14, wherein the primary modification comprises a site on the base for attachment of a primary polynucleotide to the base, and the further comprising attaching a primary polynucleotide to the base after the polymerizing comprises forming a covalent bond selected from an amine-NHS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

16. The method of claim 8, wherein the site on a base for covalent attachment of a secondary polynucleotide is a site for attachment by a covalent bond selected from an amine-NHS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

17. The method of claim 10, wherein covalently attaching a secondary polynucleotide to one or more nucleotides of the primary polynucleotides comprising a secondary modification comprises forming a covalent bond selected from an amine-NHS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.

18. The method of claim 9, further comprising attaching a secondary polynucleotide to one or more nucleotide of the primary polynucleotides comprising a secondary modification.

19. The method of claim 18, comprising covalently attaching a secondary polynucleotide to one or more nucleotide of the primary polynucleotides comprising a secondary modification.

20. The method of claim 18, wherein covalently attaching a secondary polynucleotide to one or more nucleotides of the primary polynucleotides comprising a secondary modification comprises forming a covalent bond selected from an amine-NHS ester covalent bond, an amine-imidoester covalent bond, an amine-pentofluorophenyl ester covalent bond, an amine-hydroxymethyl phosphine covalent bond, a carboxyl-carbodiimide covalent bond, a thiol-maleimide covalent bond, a thiol-haloacetyl covalent bond, a thiol-pyridyl disulfide covalent bond, a thiol-thiosulfonate covalent bond, a thiol-vinyl sulfone covalent bond, an aldehyde-hydrazide covalent bond, an aldehyde-alkoxyamine covalent bond, a hydroxy-isocyanate covalent bond, an azide-alkyne covalent bond, an azide-phosphine covalent bond, a transcyclooctene-tetrazine covalent bond, a norbornene-tetrazine covalent bond, an azide-cyclooctyne covalent bond, and an azide-norbornene covalent bond.


SEQUENCE LISTING (1 sequence)

SEQ ID NO: 1 - protein, 504 aa, Artificial Sequence
  Laboratory synthesized sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGELMRTDYSATPNPGFQKTPPLAVKKISQYACQRKTTLNNYNHIFTDAFEILAENSVFNGNEVSYVTFMRAASVLKSLPFTIISMKDTEGIPCLGDKVKCIIEEIIEYGESSEVKAVLNDERYQSFKLFTSVFGVGLKTSEKWFRMGFRSLSEIMSDKTLKLTKKQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLPHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA